(19) United States
(12) Patent Application Publication
(10) Pub. No.: US 2022/0239954 A1
(43) Pub. Date: Jul. 28, 2022

(54) Filter Flags for Subpicture Deblocking

(71) Applicant: Huawei Technologies Co., Ltd., Shenzhen (CN)

(72) Inventors: FNU Hendry, San Diego, CA (US); Ye-Kui Wang, San Diego, CA (US); Jianle Chen, San Diego, CA (US)

(21) Appl. No.: 17/698,705

(22) Filed: Mar. 18, 2022

Related U.S. Application Data
(63) Continuation of application No. PCT/US2020/052287, filed on Sep. 23, 2020.
(60) Provisional application No. 62/905,231, filed on Sep. 24, 2019.

(51) Int. Cl. H04N 19/86; H04N 19/82; H04N 19/176

(52) U.S. Cl. CPC H04N 19/86; H04N 19/176; H04N 19/82

(57) ABSTRACT

A method implemented by a video decoder and comprising: receiving, by the video decoder, a video bitstream comprising a picture and loop_filter_across_subpic_enabled_flag, wherein the picture comprises a subpicture; and applying a deblocking filter process to all subblock edges and transform block edges of the picture except edges that coincide with boundaries of the subpicture when loop_filter_across_subpic_enabled_flag is equal to 0. A method implemented by a video decoder and comprising: receiving, by the video decoder, a video bitstream comprising a picture, EDGE_VER, and loop_filter_across_subpic_enabled_flag, wherein the picture comprises a subpicture; and setting filterEdgeFlag to 0 if edgeType is equal to the EDGE_VER, a left boundary of a current coding block is a left boundary of the subpicture, and the loop_filter_across_subpic_enabled_flag is equal to 0.

### CROSS-REFERENCE TO RELATED APPLICATIONS

This is a continuation of Int'l Patent App. No. PCT/US2020/052287 filed on Sep. 23, 2020, which claims priority to U.S. Prov. Patent App. No. 62/905,231 filed on Sep. 24, 2019, both of which are incorporated by reference.

### TECHNICAL FIELD

The disclosed embodiments relate to video coding in general and filter flags for subpicture deblocking in particular.

### BACKGROUND

The amount of video data needed to depict even a relatively short video can be substantial, which may result in difficulties when the data is to be streamed or otherwise communicated across a communications network with limited bandwidth capacity. Thus, video data is generally compressed before being communicated across modern day telecommunications networks. The size of a video could also be an issue when the video is stored on a storage device because memory resources may be limited. Video compression devices often use software and/or hardware at the source to code the video data prior to transmission or storage, thereby decreasing the quantity of data needed to represent digital video images. The compressed data is then received at the destination by a video decompression device that decodes the video data. With limited network resources and ever increasing demands of higher video quality, improved compression and decompression techniques that improve compression ratio with little to no sacrifice in image quality are desirable.

### SUMMARY

A first aspect relates to a method implemented by a video decoder and comprising: receiving, by the video decoder, a video bitstream comprising a picture and loop_filter_across_subpic_enabled_flag, wherein the picture comprises a subpicture; and applying a deblocking filter process to all subblock edges and transform block edges of the picture except edges that coincide with boundaries of the subpicture when loop_filter_across_subpic_enabled_flag is equal to 0.

In a first embodiment, when two subpictures are adjacent to each other (e.g., the right boundary of the first subpicture is also the left boundary of the second subpicture or the bottom boundary of the first subpicture is also the top boundary of the second subpicture), and the values of loop_filter_across_subpic_enabled_flag[i] of the two subpictures are different, two conditions apply to deblocking of the boundary shared by the two subpictures. First, for the subpicture with loop_filter_across_subpic_enabled_flag[i] equal to 0, deblocking is not applied to the blocks at the boundary shared with the adjacent subpicture. Second, for the subpicture with loop_filter_across_subpic_enabled_flag[i] equal to 1, deblocking is applied to the blocks at the boundary shared with the adjacent subpicture. To implement that deblocking, boundary strength determination is applied per the normal deblocking process, and sample filtering is applied only to samples belonging to the subpicture with loop_filter_across_subpic_enabled_flag[i] equal to 1. In a second embodiment, when there is a subpicture with the value of subpic_treated_as_pic_flag[i] equal to 1 and loop_filter_across_subpic_enabled_flag[i] equal to 0, the value of loop_filter_across_subpic_enabled_flag[i] of all subpictures shall be equal to 0. In a third embodiment, instead of signaling loop_filter_across_subpic_enabled_flag[i] for each subpicture, only one flag is signaled to specify whether or not loop filter across subpictures is enabled. The disclosed embodiments reduce or eliminate the artifacts described above and result in fewer wasted bits in the encoded bitstream

Optionally, in any of the preceding aspects, loop_filter_across_subpic_enabled_flag equal to 1 specifies that in-loop filtering operations may be performed across boundaries of a subpicture in each coded picture in a CVS.

Optionally, in any of the preceding aspects, loop_filter_across_subpic_enabled_flag equal to 0 specifies that in-loop filtering operations are not performed across boundaries of a subpicture in each coded picture in a CVS.

A second aspect relates to A method implemented by a video encoder and comprising: generating, by the video encoder, loop_filter_across_subpic_enabled_flag so that a deblocking filter process is applied to all subblock edges and transform block edges of a picture except edges that coincide with boundaries of a subpicture when loop_filter_across_subpic_enabled_flag is equal to 0; encoding, by the video encoder, loop_filter_across_subpic_enabled_flag into a video bitstream; and storing, by the video encoder, the video bitstream for communication toward a video decoder.

Optionally, in any of the preceding aspects, loop_filter_across_subpic_enabled_flag equal to 1 specifies that in-loop filtering operations may be performed across boundaries of a subpicture in each coded picture in a CVS.

Optionally, in any of the preceding aspects, loop_filter_across_subpic_enabled_flag equal to 0 specifies that in-loop filtering operations are not performed across boundaries of a subpicture in each coded picture in a CVS.

Optionally, in any of the preceding aspects, the method further comprises generating seq_parameter_set_rbsp; including loop_filter_across_subpic_enabled_flag in seq_parameter_set_rbsp; and further encoding loop_filter_across_subpic_enabled_flag into the video bitstream by encoding seq_parameter_set_rbsp into the video bitstream.

A third aspect relates to a method implemented by a video decoder and comprising: receiving, by the video decoder, a video bitstream comprising a picture, EDGE_VER, and loop_filter_across_subpic_enabled_flag, wherein the picture comprises a subpicture; and setting filterEdgeFlag to 0 if edgeType is equal to the EDGE_VER, a left boundary of a current coding block is a left boundary of the subpicture, and the loop_filter_across_subpic_enabled_flag is equal to 0.

Optionally, in any of the preceding aspects, the edgeType is a variable specifying whether a vertical edge or a horizontal edge is filtered.

Optionally, in any of the preceding aspects, the edgeType equal to 0 specifies that the vertical edge is filtered, and wherein the EDGE_VER is the vertical edge.

Optionally, in any of the preceding aspects, the edgeType equal to 1 specifies that the horizontal edge is filtered, and wherein the EDGE_HOR is the horizontal edge.

Optionally, in any of the preceding aspects, the loop_filter_across_subpic_enabled_flag equal to 0 specifies that in-loop filtering operations are not performed across boundaries of a subpicture in each coded picture in a CVS.

Optionally, in any of the preceding aspects, the method further comprises filtering the picture based on the filterEdgeFlag.

A fourth aspect relates to a method implemented by a video decoder and comprising: receiving, by the video decoder, a video bitstream comprising a picture, EDGE_HOR, and loop_filter_across_subpic_enabled_flag, wherein the picture comprises a subpicture; and setting filterEdgeFlag to 0 if edgeType is equal to the EDGE_HOR, a top boundary of a current coding block is a top boundary of the subpicture, and the loop_filter_across_subpic_enabled_flag is equal to 0.

Optionally, in any of the preceding aspects, the edgeType is a variable specifying whether a vertical edge or a horizontal edge is filtered.

Optionally, in any of the preceding aspects, the edgeType equal to 0 specifies that the vertical edge is filtered, and wherein the EDGE_VER is the vertical edge.

Optionally, in any of the preceding aspects, the edgeType equal to 1 specifies that the horizontal edge is filtered, and wherein the EDGE_HOR is the horizontal edge.

Optionally, in any of the preceding aspects, the loop_filter_across_subpic_enabled_flag equal to 0 specifies that in-loop filtering operations are not performed across boundaries of a subpicture in each coded picture in a CVS.

Optionally, in any of the preceding aspects, the method further comprises filtering the picture based on the filterEdgeFlag.

A fifth aspect relates to a method implemented by a video decoder and comprising: receiving, by the video decoder, a video bitstream comprising a picture and loop_filter_across_subpic_enabled_flag, wherein the picture comprises a subpicture; and applying an SAO process to all subblock edges and transform block edges of the picture except edges that coincide with boundaries of the subpicture when loop_filter_across_subpic_enabled_flag is equal to 0.

A sixth aspect relates to a method implemented by a video decoder and comprising: receiving, by the video decoder, a video bitstream comprising a picture and loop_filter_across_subpic_enabled_flag, wherein the picture comprises a subpicture; and applying an ALF process to all subblock edges and transform block edges of the picture except edges that coincide with boundaries of the subpicture when loop_filter_across_subpic_enabled_flag is equal to 0.

Any of the above embodiments may be combined with any of the other above embodiments to create a new embodiment. These and other features will be more clearly understood from the following detailed description taken in conjunction with the accompanying drawings and claims.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a flowchart of an example method of coding a video signal.

FIG. 2 is a schematic diagram of an example coding and decoding (codec) system for video coding.

FIG. 3 is a schematic diagram illustrating an example video encoder.

FIG. 4 is a schematic diagram illustrating an example video decoder.

FIG. 5 is a schematic diagram illustrating a plurality of sub-picture video streams extracted from a picture video stream.

FIG. 6 is a schematic diagram illustrating an example bitstream split into a sub-bitstream.

FIG. 7 is a flowchart illustrating a method of decoding a bitstream according to a first embodiment.

FIG. 8 is a flowchart illustrating a method of encoding a bitstream according to a first embodiment.

FIG. 9 is a flowchart illustrating a method of decoding a bitstream according to a second embodiment.

FIG. 10 is a flowchart illustrating a method of decoding a bitstream according to a third embodiment.

FIG. 11 is a schematic diagram of a video coding device.

FIG. 12 is a schematic diagram of an embodiment of a means for coding.

### DETAILED DESCRIPTION

It should be understood at the outset that, although an illustrative implementation of one or more embodiments are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

The following abbreviations apply:

ALF: adaptive loop filter

ASIC: application-specific integrated circuit

AU: access unit

AUD: access unit delimiter

BT: binary tree

CABAC: context-adaptive binary arithmetic coding

CAVLC: context-adaptive variable-length coding

Cb: blue difference chroma

CPU: central processing unit

Cr: red difference chroma

CTB: coding tree block

CTU: coding tree unit

CU: coding unit

CVS: coded video sequence

DC: direct current

DCT: discrete cosine transform

DMM: depth modeling mode

DPB: decoded picture buffer

DSP: digital signal processor

DST: discrete sine transform

EO: electrical-to-optical

FPGA: field-programmable gate array

HEVC: High Efficiency Video Coding

HMD: head-mounted display

I/O: input/output

NAL: network abstraction layer

OE: optical-to-electrical

PIPE: probability interval partitioning entropy

POC: picture order count

PPS: picture parameter set

PU: picture unit

QT: quad tree

RAM: random-access memory

RBSP: raw byte sequence payload

RDO: rate-distortion optimization

ROM: read-only memory

RPL: reference picture list

Rx: receiver unit

SAD: sum of absolute differences

SAO: sample adaptive offset

SBAC: syntax-based arithmetic coding

SPS: sequence parameter set

SRAM: static RAM

SSD: sum of squared differences

TCAM: ternary content-addressable memory

TT: triple tree

TU: transform unit

Tx: transmitter unit

VR: virtual reality

VVC: Versatile Video Coding.

The following definitions apply unless modified elsewhere: A bitstream is a sequence of bits including video data that is compressed for transmission between an encoder and a decoder. An encoder is a device that employs encoding processes to compress video data into a bitstream. A decoder is a device that employs decoding processes to reconstruct video data from a bitstream for display. A picture is an array of luma samples or chroma samples that creates a frame or a field. A picture that is being encoded or decoded can be referred to as a current picture. A reference picture contains reference samples that can be used when coding other pictures by reference according to inter-prediction or inter-layer prediction. A reference picture list is a list of reference pictures used for inter-prediction or inter-layer prediction. A flag is a variable or single-bit syntax element that can take one of the two possible values: 0 or 1. Some video coding systems utilize two reference picture lists, which can be denoted as reference picture list one and reference picture list zero. A reference picture list structure is an addressable syntax structure that contains multiple reference picture lists. Inter-prediction is a mechanism of coding samples of a current picture by reference to indicated samples in a reference picture that is different from the current picture, where the reference picture and the current picture are in the same layer. A reference picture list structure entry is an addressable location in a reference picture list structure that indicates a reference picture associated with a reference picture list. A slice header is a part of a coded slice containing data elements pertaining to all video data within a tile represented in the slice. A PPS contains data related to an entire picture. More specifically, the PPS is a syntax structure containing syntax elements that apply to zero or more entire coded pictures as determined by a syntax element found in each picture header. An SPS contains data related to a sequence of pictures. An AU is a set of one or more coded pictures associated with the same display time (e.g., the same picture order count) for output from a DPB (e.g., for display to a user). An AUD indicates the start of an AU or the boundary between AUs. A decoded video sequence is a sequence of pictures that have been reconstructed by a decoder in preparation for display to a user.

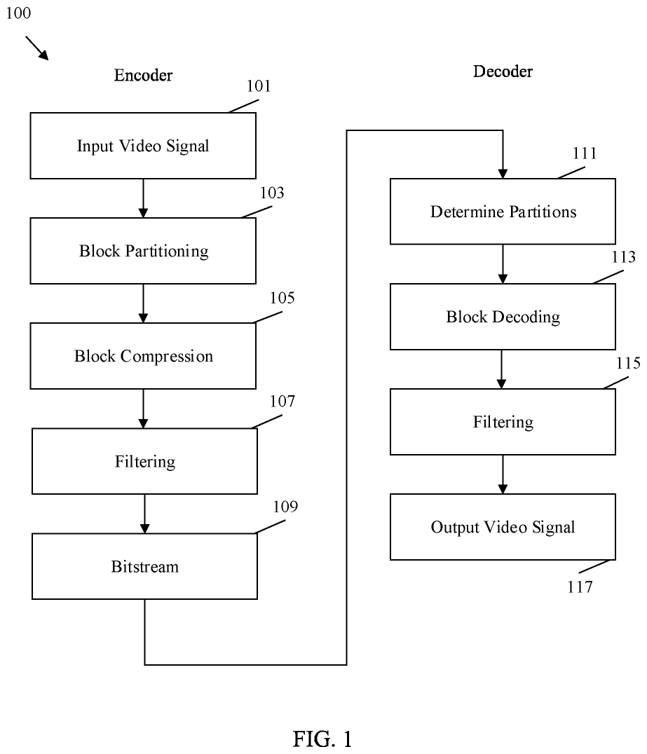

FIG. 1 is a flowchart of an example operating method 100 of coding a video signal. Specifically, a video signal is encoded at an encoder. The encoding process compresses the video signal by employing various mechanisms to reduce the video file size. A smaller file size allows the compressed video file to be transmitted toward a user, while reducing associated bandwidth overhead. The decoder then decodes the compressed video file to reconstruct the original video signal for display to an end user. The decoding process generally mirrors the encoding process to allow the decoder to consistently reconstruct the video signal.

At step 101, the video signal is input into the encoder. For example, the video signal may be an uncompressed video file stored in memory. As another example, the video file may be captured by a video capture device, such as a video camera, and encoded to support live streaming of the video. The video file may include both an audio component and a video component. The video component contains a series of image frames that, when viewed in a sequence, gives the visual impression of motion. The frames contain pixels that are expressed in terms of light, referred to herein as luma components (or luma samples), and color, which is referred to as chroma components (or color samples). In some examples, the frames may also contain depth values to support three dimensional viewing.

At step 103, the video is partitioned into blocks. Partitioning includes subdividing the pixels in each frame into square and/or rectangular blocks for compression. For example, in HEVC, the frame can first be divided into CTUs, which are blocks of a predefined size (e.g., sixty-four pixels by sixty-four pixels). The CTUs contain both luma and chroma samples. Coding trees may be employed to divide the CTUs into blocks and then recursively subdivide the blocks until configurations are achieved that support further encoding. For example, luma components of a frame may be subdivided until the individual blocks contain relatively homogenous lighting values. Further, chroma components of a frame may be subdivided until the individual blocks contain relatively homogenous color values. Accordingly, partitioning mechanisms vary depending on the content of the video frames.

At step 105, various compression mechanisms are employed to compress the image blocks partitioned at step 103. For example, inter-prediction and/or intra-prediction may be employed. Inter-prediction is designed to take advantage of the fact that objects in a common scene tend to appear in successive frames. Accordingly, a block depicting an object in a reference frame need not be repeatedly described in adjacent frames. Specifically, an object, such as a table, may remain in a constant position over multiple frames. Hence the table is described once and adjacent frames can refer back to the reference frame. Pattern matching mechanisms may be employed to match objects over multiple frames. Further, moving objects may be represented across multiple frames, for example due to object movement or camera movement. As a particular example, a video may show an automobile that moves across the screen over multiple frames. Motion vectors can be employed to describe such movement. A motion vector is a two-dimensional vector that provides an offset from the coordinates of an object in a frame to the coordinates of the object in a reference frame. As such, inter-prediction can encode an image block in a current frame as a set of motion vectors indicating an offset from a corresponding block in a reference frame.

Intra-prediction encodes blocks in a common frame. Intra-prediction takes advantage of the fact that luma and chroma components tend to cluster in a frame. For example, a patch of green in a portion of a tree tends to be positioned adjacent to similar patches of green. Intra-prediction employs multiple directional prediction modes (e.g., 33 in HEVC), a planar mode, and a DC mode. The directional modes indicate that a current block is similar/the same as samples of a neighbor block in a corresponding direction. Planar mode indicates that a series of blocks along a row/column (e.g., a plane) can be interpolated based on neighbor blocks at the edges of the row. Planar mode, in effect, indicates a smooth transition of light/color across a row/column by employing a relatively constant slope in changing values. DC mode is employed for boundary smoothing and indicates that a block is similar/the same as an average value associated with samples of all the neighbor blocks associated with the angular directions of the directional prediction modes. Accordingly, intra-prediction blocks can represent image blocks as various relational prediction mode values instead of the actual values. Further, inter-prediction blocks can represent image blocks as motion vector values instead of the actual values. In either case, the prediction blocks may not exactly represent the image blocks in some cases. Any differences are stored in residual blocks. Transforms may be applied to the residual blocks to further compress the file.

At step 107, various filtering techniques may be applied. In HEVC, the filters are applied according to an in-loop filtering scheme. The block based prediction discussed above may result in the creation of blocky images at the decoder. Further, the block based prediction scheme may encode a block and then reconstruct the encoded block for later use as a reference block. The in-loop filtering scheme iteratively applies noise suppression filters, de-blocking filters, adaptive loop filters, and SAO filters to the blocks/frames. These filters mitigate such blocking artifacts so that the encoded file can be accurately reconstructed. Further, these filters mitigate artifacts in the reconstructed reference blocks so that artifacts are less likely to create additional artifacts in subsequent blocks that are encoded based on the reconstructed reference blocks.

Once the video signal has been partitioned, compressed, and filtered, the resulting data is encoded in a bitstream at step 109. The bitstream includes the data discussed above as well as any signaling data desired to support proper video signal reconstruction at the decoder. For example, such data may include partition data, prediction data, residual blocks, and various flags providing coding instructions to the decoder. The bitstream may be stored in memory for transmission toward a decoder upon request. The bitstream may also be broadcast and/or multicast toward a plurality of decoders. The creation of the bitstream is an iterative process. Accordingly, steps 101, 103, 105, 107, and 109 may occur continuously and/or simultaneously over many frames and blocks. The order shown in FIG. 1 is presented for clarity and ease of discussion, and is not intended to limit the video coding process to a particular order.

The decoder receives the bitstream and begins the decoding process at step 111. Specifically, the decoder employs an entropy decoding scheme to convert the bitstream into corresponding syntax and video data. The decoder employs the syntax data from the bitstream to determine the partitions for the frames at step 111. The partitioning should match the results of block partitioning at step 103. Entropy encoding/decoding as employed in step 111 is now described. The encoder makes many choices during the compression process, such as selecting block partitioning schemes from several possible choices based on the spatial positioning of values in the input image(s). Signaling the exact choices may employ a large number of bins. As used herein, a bin is a binary value that is treated as a variable (e.g., a bit value that may vary depending on context). Entropy coding allows the encoder to discard any options that are clearly not viable for a particular case, leaving a set of allowable options. Each allowable option is then assigned a code word. The length of the code words is based on the number of allowable options (e.g., one bin for two options, two bins for three to four options, etc.) The encoder then encodes the code word for the selected option. This scheme reduces the size of the code words as the code words are as big as desired to uniquely indicate a selection from a small sub-set of allowable options as opposed to uniquely indicating the selection from a potentially large set of all possible options. The decoder then decodes the selection by determining the set of allowable options in a similar manner to the encoder. By determining the set of allowable options, the decoder can read the code word and determine the selection made by the encoder.

At step 113, the decoder performs block decoding. Specifically, the decoder employs reverse transforms to generate residual blocks. Then the decoder employs the residual blocks and corresponding prediction blocks to reconstruct the image blocks according to the partitioning. The prediction blocks may include both intra-prediction blocks and inter-prediction blocks as generated at the encoder at step 105. The reconstructed image blocks are then positioned into frames of a reconstructed video signal according to the partitioning data determined at step 111. Syntax for step 113 may also be signaled in the bitstream via entropy coding as discussed above.

At step 115, filtering is performed on the frames of the reconstructed video signal in a manner similar to step 107 at the encoder. For example, noise suppression filters, de-blocking filters, adaptive loop filters, and SAO filters may be applied to the frames to remove blocking artifacts. Once the frames are filtered, the video signal can be output to a display at step 117 for viewing by an end user.

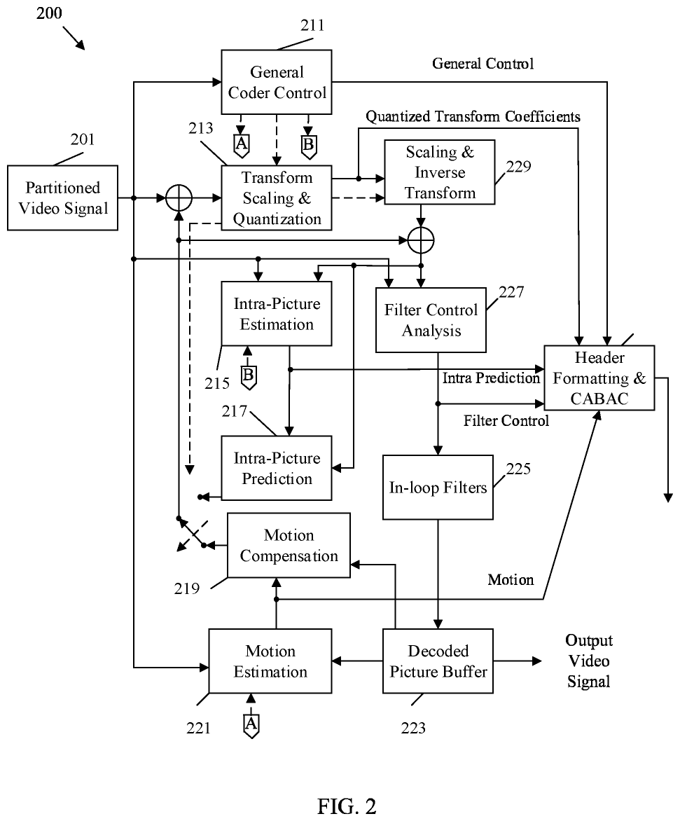

FIG. 2 is a schematic diagram of an example coding and decoding (codec) system 200 for video coding. Specifically, codec system 200 provides functionality to support the implementation of operating method 100. Codec system 200 is generalized to depict components employed in both an encoder and a decoder. Codec system 200 receives and partitions a video signal as discussed with respect to steps 101 and 103 in operating method 100, which results in a partitioned video signal 201. Codec system 200 then compresses the partitioned video signal 201 into a coded bitstream when acting as an encoder as discussed with respect to steps 105, 107, and 109 in method 100. When acting as a decoder, codec system 200 generates an output video signal from the bitstream as discussed with respect to steps 111, 113, 115, and 117 in operating method 100. The codec system 200 includes a general coder control component 211, a transform scaling and quantization component 213, an intra-picture estimation component 215, an intra-picture prediction component 217, a motion compensation component 219, a motion estimation component 221, a scaling and inverse transform component 229, a filter control analysis component 227, an in-loop filters component 225, a decoded picture buffer component 223, and a header formatting and CABAC component 231. Such components are coupled as shown. In FIG. 2, black lines indicate movement of data to be encoded/decoded while dashed lines indicate movement of control data that controls the operation of other components. The components of codec system 200 may all be present in the encoder. The decoder may include a subset of the components of codec system 200. For example, the decoder may include the intra-picture prediction component 217, the motion compensation component 219, the scaling and inverse transform component 229, the in-loop filters component 225, and the decoded picture buffer component 223. These components are now described.

The partitioned video signal 201 is a captured video sequence that has been partitioned into blocks of pixels by a coding tree. A coding tree employs various split modes to subdivide a block of pixels into smaller blocks of pixels. These blocks can then be further subdivided into smaller blocks. The blocks may be referred to as nodes on the coding tree. Larger parent nodes are split into smaller child nodes. The number of times a node is subdivided is referred to as the depth of the node/coding tree. The divided blocks can be included in CUs in some cases. For example, a CU can be a sub-portion of a CTU that contains a luma block, Cr block(s), and a Cb block(s) along with corresponding syntax instructions for the CU. The split modes may include a BT, TT, and QT employed to partition a node into two, three, or four child nodes, respectively, of varying shapes depending on the split modes employed. The partitioned video signal 201 is forwarded to the general coder control component 211, the transform scaling and quantization component 213, the intra-picture estimation component 215, the filter control analysis component 227, and the motion estimation component 221 for compression.

The general coder control component 211 is configured to make decisions related to coding of the images of the video sequence into the bitstream according to application constraints. For example, the general coder control component 211 manages optimization of bitrate/bitstream size versus reconstruction quality. Such decisions may be made based on storage space/bandwidth availability and image resolution requests. The general coder control component 211 also manages buffer utilization in light of transmission speed to mitigate buffer underrun and overrun issues. To manage these issues, the general coder control component 211 manages partitioning, prediction, and filtering by the other components. For example, the general coder control component 211 may dynamically increase compression complexity to increase resolution and increase bandwidth usage or decrease compression complexity to decrease resolution and bandwidth usage. Hence, the general coder control component 211 controls the other components of codec system 200 to balance video signal reconstruction quality with bit rate concerns. The general coder control component 211 creates control data, which controls the operation of the other components. The control data is also forwarded to the header formatting and CABAC component 231 to be encoded in the bitstream to signal parameters for decoding at the decoder.

The partitioned video signal 201 is also sent to the motion estimation component 221 and the motion compensation component 219 for inter-prediction. A frame or slice of the partitioned video signal 201 may be divided into multiple video blocks. Motion estimation component 221 and the motion compensation component 219 perform inter-predictive coding of the received video block relative to one or more blocks in one or more reference frames to provide temporal prediction. Codec system 200 may perform multiple coding passes, e.g., to select an appropriate coding mode for each block of video data.

Motion estimation component 221 and motion compensation component 219 may be highly integrated, but are illustrated separately for conceptual purposes. Motion estimation, performed by motion estimation component 221, is the process of generating motion vectors, which estimate motion for video blocks. A motion vector, for example, may indicate the displacement of a coded object relative to a predictive block. A predictive block is a block that is found to closely match the block to be coded, in terms of pixel difference. A predictive block may also be referred to as a reference block. Such pixel difference may be determined by an SAD, an SSD, or other difference metrics. HEVC employs several coded objects including a CTU, CTBs, and CUs. For example, a CTU can be divided into CTBs, which can then be divided into CBs for inclusion in CUs. A CU can be encoded as a prediction unit containing prediction data and/or a TU containing transformed residual data for the CU. The motion estimation component 221 generates motion vectors, prediction units, and TUs by using a rate-distortion analysis as part of a rate distortion optimization process. For example, the motion estimation component 221 may determine multiple reference blocks, multiple motion vectors, etc. for a current block/frame, and may select the reference blocks, motion vectors, etc. having the best rate-distortion characteristics. The best rate-distortion characteristics balance both quality of video reconstruction (e.g., amount of data loss by compression) with coding efficiency (e.g., size of the final encoding).

In some examples, codec system 200 may calculate values for sub-integer pixel positions of reference pictures stored in decoded picture buffer component 223. For example, video codec system 200 may interpolate values of one-quarter pixel positions, one-eighth pixel positions, or other fractional pixel positions of the reference picture. Therefore, motion estimation component 221 may perform a motion search relative to the full pixel positions and fractional pixel positions and output a motion vector with fractional pixel precision. The motion estimation component 221 calculates a motion vector for a prediction unit of a video block in an inter-coded slice by comparing the position of the prediction unit to the position of a predictive block of a reference picture. Motion estimation component 221 outputs the calculated motion vector as motion data to header formatting and CABAC component 231 for encoding and motion to the motion compensation component 219.

Motion compensation, performed by motion compensation component 219, may involve fetching or generating the predictive block based on the motion vector determined by motion estimation component 221. Again, motion estimation component 221 and motion compensation component 219 may be functionally integrated, in some examples. Upon receiving the motion vector for the prediction unit of the current video block, motion compensation component 219 may locate the predictive block to which the motion vector points. A residual video block is then formed by subtracting pixel values of the predictive block from the pixel values of the current video block being coded, forming pixel difference values. In general, motion estimation component 221 performs motion estimation relative to luma components, and motion compensation component 219 uses motion vectors calculated based on the luma components for both chroma components and luma components. The predictive block and residual block are forwarded to transform scaling and quantization component 213.

The partitioned video signal 201 is also sent to intra-picture estimation component 215 and intra-picture prediction component 217. As with motion estimation component 221 and motion compensation component 219, intra-picture estimation component 215 and intra-picture prediction component 217 may be highly integrated, but are illustrated separately for conceptual purposes. The intra-picture estimation component 215 and intra-picture prediction component 217 intra-predict a current block relative to blocks in a current frame, as an alternative to the inter-prediction performed by motion estimation component 221 and motion compensation component 219 between frames, as described above. In particular, the intra-picture estimation component 215 determines an intra-prediction mode to use to encode a current block. In some examples, intra-picture estimation component 215 selects an appropriate intra-prediction mode to encode a current block from multiple tested intra-prediction modes. The selected intra-prediction modes are then forwarded to the header formatting and CABAC component 231 for encoding.

For example, the intra-picture estimation component 215 calculates rate-distortion values using a rate-distortion analysis for the various tested intra-prediction modes, and selects the intra-prediction mode having the best rate-distortion characteristics among the tested modes. Rate-distortion analysis generally determines an amount of distortion (or error) between an encoded block and an original un-encoded block that was encoded to produce the encoded block, as well as a bitrate (e.g., a number of bits) used to produce the encoded block. The intra-picture estimation component 215 calculates ratios from the distortions and rates for the various encoded blocks to determine which intra-prediction mode exhibits the best rate-distortion value for the block. In addition, intra-picture estimation component 215 may be configured to code depth blocks of a depth map using a DMM based on RDO.

The intra-picture prediction component 217 may generate a residual block from the predictive block based on the selected intra-prediction modes determined by intra-picture estimation component 215 when implemented on an encoder or read the residual block from the bitstream when implemented on a decoder. The residual block includes the difference in values between the predictive block and the original block, represented as a matrix. The residual block is then forwarded to the transform scaling and quantization component 213. The intra-picture estimation component 215 and the intra-picture prediction component 217 may operate on both luma and chroma components.

The transform scaling and quantization component 213 is configured to further compress the residual block. The transform scaling and quantization component 213 applies a transform, such as a DCT, a DST, or a conceptually similar transform, to the residual block, producing a video block comprising residual transform coefficient values. Wavelet transforms, integer transforms, sub-band transforms or other types of transforms could also be used. The transform may convert the residual information from a pixel value domain to a transform domain, such as a frequency domain. The transform scaling and quantization component 213 is also configured to scale the transformed residual information, for example based on frequency. Such scaling involves applying a scale factor to the residual information so that different frequency information is quantized at different granularities, which may affect final visual quality of the reconstructed video. The transform scaling and quantization component 213 is also configured to quantize the transform coefficients to further reduce bit rate. The quantization process may reduce the bit depth associated with some or all of the coefficients. The degree of quantization may be modified by adjusting a quantization parameter. In some examples, the transform scaling and quantization component 213 may then perform a scan of the matrix including the quantized transform coefficients. The quantized transform coefficients are forwarded to the header formatting and CABAC component 231 to be encoded in the bitstream.

The scaling and inverse transform component 229 applies a reverse operation of the transform scaling and quantization component 213 to support motion estimation. The scaling and inverse transform component 229 applies inverse scaling, transformation, and/or quantization to reconstruct the residual block in the pixel domain, e.g., for later use as a reference block which may become a predictive block for another current block. The motion estimation component 221 and/or motion compensation component 219 may calculate a reference block by adding the residual block back to a corresponding predictive block for use in motion estimation of a later block/frame. Filters are applied to the reconstructed reference blocks to mitigate artifacts created during scaling, quantization, and transform. Such artifacts could otherwise cause inaccurate prediction (and create additional artifacts) when subsequent blocks are predicted.

The filter control analysis component 227 and the in-loop filters component 225 apply the filters to the residual blocks and/or to reconstructed image blocks. For example, the transformed residual block from the scaling and inverse transform component 229 may be combined with a corresponding prediction block from intra-picture prediction component 217 and/or motion compensation component 219 to reconstruct the original image block. The filters may then be applied to the reconstructed image block. In some examples, the filters may instead be applied to the residual blocks. As with other components in FIG. 2, the filter control analysis component 227 and the in-loop filters component 225 are highly integrated and may be implemented together, but are depicted separately for conceptual purposes. Filters applied to the reconstructed reference blocks are applied to particular spatial regions and include multiple parameters to adjust how such filters are applied. The filter control analysis component 227 analyzes the reconstructed reference blocks to determine where such filters should be applied and sets corresponding parameters. Such data is forwarded to the header formatting and CABAC component 231 as filter control data for encoding. The in-loop filters component 225 applies such filters based on the filter control data. The filters may include a de-blocking filter, a noise suppression filter, an SAO filter, and an adaptive loop filter. Such filters may be applied in the spatial/pixel domain (e.g., on a reconstructed pixel block) or in the frequency domain, depending on the example.

When operating as an encoder, the filtered reconstructed image block, residual block, and/or prediction block are stored in the decoded picture buffer component 223 for later use in motion estimation as discussed above. When operating as a decoder, the decoded picture buffer component 223 stores and forwards the reconstructed and filtered blocks toward a display as part of an output video signal. The decoded picture buffer component 223 may be any memory device capable of storing prediction blocks, residual blocks, and/or reconstructed image blocks.

The header formatting and CABAC component 231 receives the data from the various components of codec system 200 and encodes such data into a coded bitstream for transmission toward a decoder. Specifically, the header formatting and CABAC component 231 generates various headers to encode control data, such as general control data and filter control data. Further, prediction data, including intra-prediction and motion data, as well as residual data in the form of quantized transform coefficient data are all encoded in the bitstream. The final bitstream includes all information desired by the decoder to reconstruct the original partitioned video signal 201. Such information may also include intra-prediction mode index tables (also referred to as codeword mapping tables), definitions of encoding contexts for various blocks, indications of most probable intra-prediction modes, an indication of partition information, etc. Such data may be encoded by employing entropy coding. For example, the information may be encoded by employing CAVLC, CABAC, SBAC, PIPE coding, or another entropy coding technique. Following the entropy coding, the coded bitstream may be transmitted to another device (e.g., a video decoder) or archived for later transmission or retrieval.

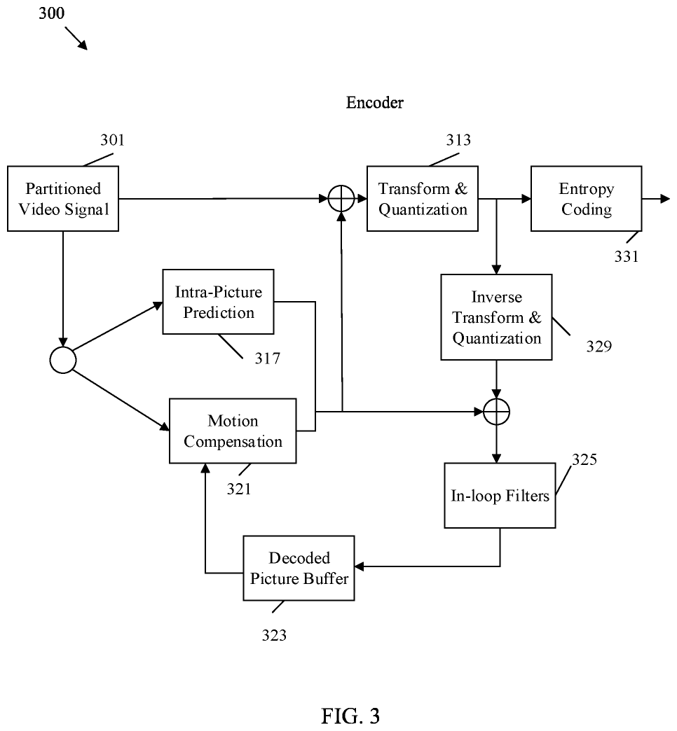

FIG. 3 is a block diagram illustrating an example video encoder 300. Video encoder 300 may be employed to implement the encoding functions of codec system 200 and/or implement steps 101, 103, 105, 107, and/or 109 of operating method 100. Encoder 300 partitions an input video signal, resulting in a partitioned video signal 301, which is substantially similar to the partitioned video signal 201. The partitioned video signal 301 is then compressed and encoded into a bitstream by components of encoder 300.

Specifically, the partitioned video signal 301 is forwarded to an intra-picture prediction component 317 for intra-prediction. The intra-picture prediction component 317 may be substantially similar to intra-picture estimation component 215 and intra-picture prediction component 217. The partitioned video signal 301 is also forwarded to a motion compensation component 321 for inter-prediction based on reference blocks in a decoded picture buffer component 323. The motion compensation component 321 may be substantially similar to motion estimation component 221 and motion compensation component 219. The prediction blocks and residual blocks from the intra-picture prediction component 317 and the motion compensation component 321 are forwarded to a transform and quantization component 313 for transform and quantization of the residual blocks. The transform and quantization component 313 may be substantially similar to the transform scaling and quantization component 213. The transformed and quantized residual blocks and the corresponding prediction blocks (along with associated control data) are forwarded to an entropy coding component 331 for coding into a bitstream. The entropy coding component 331 may be substantially similar to the header formatting and CABAC component 231.

The transformed and quantized residual blocks and/or the corresponding prediction blocks are also forwarded from the transform and quantization component 313 to an inverse transform and quantization component 329 for reconstruction into reference blocks for use by the motion compensation component 321. The inverse transform and quantization component 329 may be substantially similar to the scaling and inverse transform component 229. In-loop filters in an in-loop filters component 325 are also applied to the residual blocks and/or reconstructed reference blocks, depending on the example. The in-loop filters component 325 may be substantially similar to the filter control analysis component 227 and the in-loop filters component 225. The in-loop filters component 325 may include multiple filters as discussed with respect to in-loop filters component 225. The filtered blocks are then stored in a decoded picture buffer component 323 for use as reference blocks by the motion compensation component 321. The decoded picture buffer component 323 may be substantially similar to the decoded picture buffer component 223.

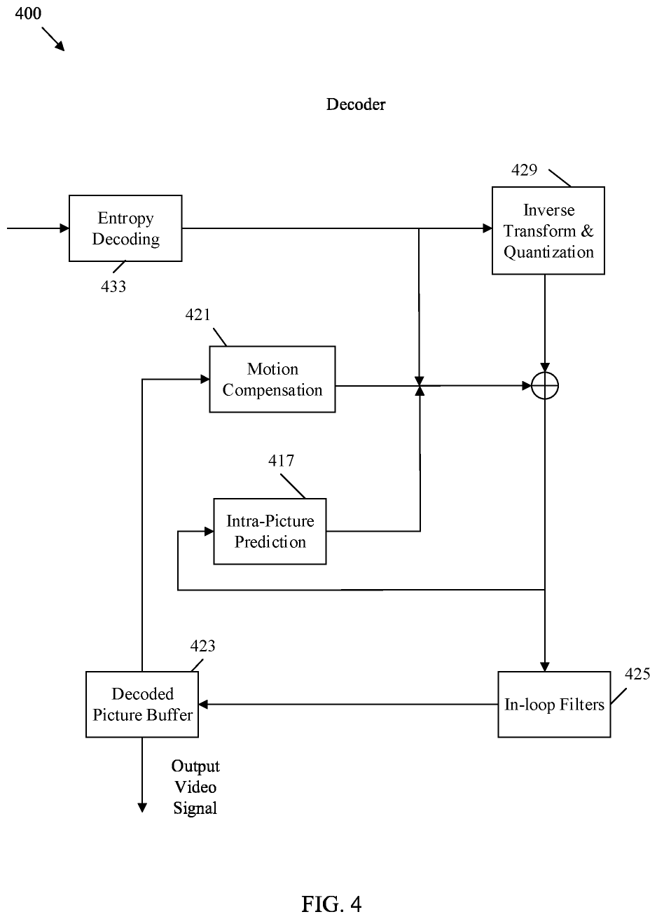

FIG. 4 is a block diagram illustrating an example video decoder 400. Video decoder 400 may be employed to implement the decoding functions of codec system 200 and/or implement steps 111, 113, 115, and/or 117 of operating method 100. Decoder 400 receives a bitstream, for example from an encoder 300, and generates a reconstructed output video signal based on the bitstream for display to an end user.

The bitstream is received by an entropy decoding component 433. The entropy decoding component 433 is configured to implement an entropy decoding scheme, such as CAVLC, CABAC, SBAC, PIPE coding, or other entropy coding techniques. For example, the entropy decoding component 433 may employ header information to provide a context to interpret additional data encoded as codewords in the bitstream. The decoded information includes any desired information to decode the video signal, such as general control data, filter control data, partition information, motion data, prediction data, and quantized transform coefficients from residual blocks. The quantized transform coefficients are forwarded to an inverse transform and quantization component 429 for reconstruction into residual blocks. The inverse transform and quantization component 429 may be similar to inverse transform and quantization component 329.

The reconstructed residual blocks and/or prediction blocks are forwarded to intra-picture prediction component 417 for reconstruction into image blocks based on intra-prediction operations. The intra-picture prediction component 417 may be similar to intra-picture estimation component 215 and an intra-picture prediction component 217. Specifically, the intra-picture prediction component 417 employs prediction modes to locate a reference block in the frame and applies a residual block to the result to reconstruct intra-predicted image blocks. The reconstructed intra-predicted image blocks and/or the residual blocks and corresponding inter-prediction data are forwarded to a decoded picture buffer component 423 via an in-loop filters component 425, which may be substantially similar to decoded picture buffer component 223 and in-loop filters component 225, respectively. The in-loop filters component 425 filters the reconstructed image blocks, residual blocks and/or prediction blocks, and such information is stored in the decoded picture buffer component 423. Reconstructed image blocks from decoded picture buffer component 423 are forwarded to a motion compensation component 421 for inter-prediction. The motion compensation component 421 may be substantially similar to motion estimation component 221 and/or motion compensation component 219. Specifically, the motion compensation component 421 employs motion vectors from a reference block to generate a prediction block and applies a residual block to the result to reconstruct an image block. The resulting reconstructed blocks may also be forwarded via the in-loop filters component 425 to the decoded picture buffer component 423. The decoded picture buffer component 423 continues to store additional reconstructed image blocks, which can be reconstructed into frames via the partition information. Such frames may also be placed in a sequence. The sequence is output toward a display as a reconstructed output video signal.

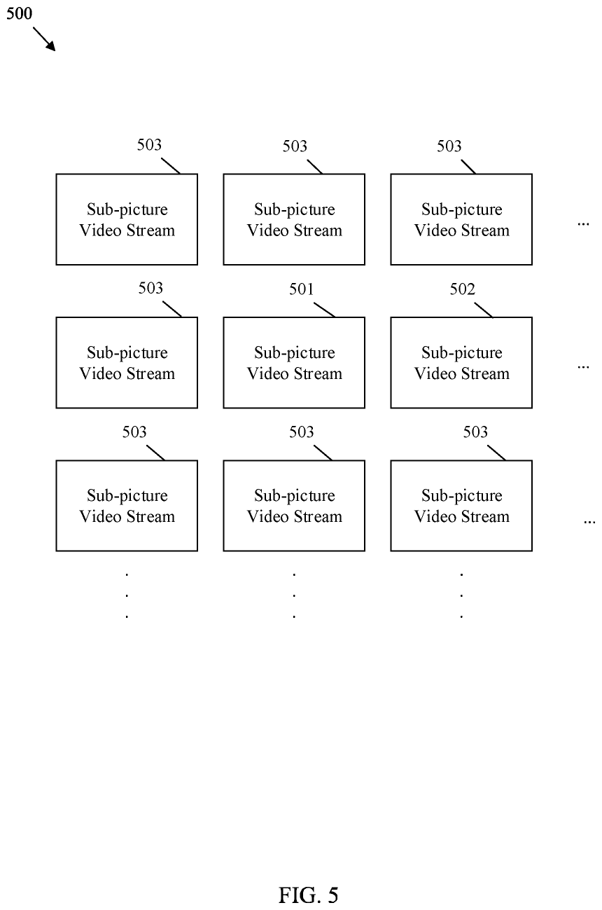

FIG. 5 is a schematic diagram illustrating a plurality of sub-picture video streams 501, 502, and 503 extracted from a picture video stream 500. For example, each of the sub-picture video streams 501-503 or the picture video stream 500 may be encoded by an encoder, such as codec system 200 or encoder 300 according to method 100. Further, the sub-picture video streams 501-503 or the picture video stream 500 may be decoded by a decoder such as codec system 200 or decoder 400.

The picture video stream 500 includes a plurality of pictures presented over time. The picture video stream 500 is configured for use in VR applications. VR operates by coding a sphere of video content, which can be displayed as if the user is in the center of the sphere. Each picture includes the entire sphere. Meanwhile, only a portion of the picture, known as a viewport, is displayed to the user. For example, the user may employ an HMD that selects and displays a viewport of the sphere based on the user's head movement. This provides the impression of being physically present in a virtual space as depicted by the video. In order to accomplish this result, each picture of the video sequence includes an entire sphere of video data at a corresponding instant in time. However, only a small portion (e.g., a single viewport) of the picture is displayed to the user. The remainder of the picture is discarded at the decoder without being rendered. The entire picture may be transmitted so that a different viewport can be dynamically selected and displayed in response to the user's head movement.

The pictures of the picture video stream 500 can each be sub-divided into sub-pictures based on available viewports. Accordingly, each picture and corresponding sub-picture includes a temporal position (e.g., picture order) as part of the temporal presentation. Sub-picture video streams 501-503 are created when the sub-division is applied consistently over time. Such consistent sub-division creates sub-picture video streams 501-503 where each stream contains a set of sub-pictures of a predetermined size, shape, and spatial position relative to corresponding pictures in the picture video stream 500. Further, the set of sub-pictures in a sub-picture video stream 501-503 varies in temporal position over the presentation time. As such, the sub-pictures of the sub-picture video streams 501-503 can be aligned in the time domain based on temporal position. Then the sub-pictures from the sub-picture video streams 501-503 at each temporal position can be merged in the spatial domain based on predefined spatial position to reconstruct the picture video stream 500 for display. Specifically, the sub-picture video streams 501-503 can each be encoded into separate sub-bitstreams. When such sub-bitstreams are merged together, they result in a bitstream that includes the entire set of pictures over time. The resulting bitstream can be transmitted toward the decoder for decoding and display based on the user's currently-selected viewport.

All of the sub-picture video streams 501-503 may be transmitted to a user at a high quality. This allows the decoder to dynamically select the user's current viewport and display the sub-pictures from the corresponding sub-picture video streams 501-503 in real time. However, the user may view only a single viewport, for example from sub-picture video stream 501, while sub-picture video streams 502-503 are discarded. As such, transmitting sub-picture video streams 502-503 at a high quality may waste a significant amount of bandwidth. In order to improve coding efficiency, the VR video may be encoded into a plurality of video streams 500 where each video stream 500 is encoded at a different quality. In this way, the decoder can transmit a request for a current sub-picture video stream 501. In response, the encoder can select the higher-quality sub-picture video stream 501 from the higher-quality video stream 500 and the lower-quality sub-picture video streams 502-503 from the lower-quality video stream 500. The encoder can then merge such sub-bitstreams together into a complete encoded bitstream for transmission to the decoder. In this way, the decoder receives a series of pictures where the current viewport is higher quality and the other viewports are lower quality. Further, the highest-quality sub-pictures are generally displayed to the user and the lower-quality sub-pictures are generally discarded, which balances functionality with coding efficiency.

In the event that the user turns from viewing the sub-picture video stream 501 to the sub-picture video stream 502, the decoder requests the new current sub-picture video stream 502 be transmitted at the higher quality. The encoder can then alter the merging mechanism accordingly.

Sub-pictures may also be employed in teleconferencing systems. In such a case, each user's video feed is included in a sub-picture bitstream, such as sub-picture video stream 501, 502, or 503. The system can receive such sub-picture video stream 501, 502, or 503 and combine them in different positions or resolutions to create a complete picture video stream 500 for transmission back to the users. This allows the teleconferencing system to dynamically change the picture video stream 500 based on changing user input, for example by increasing or decreasing the size of a sub-picture video stream 501, 502, or 503, to emphasize users who are currently speaking or deemphasize users who are no longer speaking. Accordingly, sub-pictures have many applications that allow a picture video stream 500 to be dynamically altered at run-time based on changes in user behavior. This functionality may be achieved by extracting or combining sub-picture video stream 501, 502, or 503 from or into the picture video stream 500.

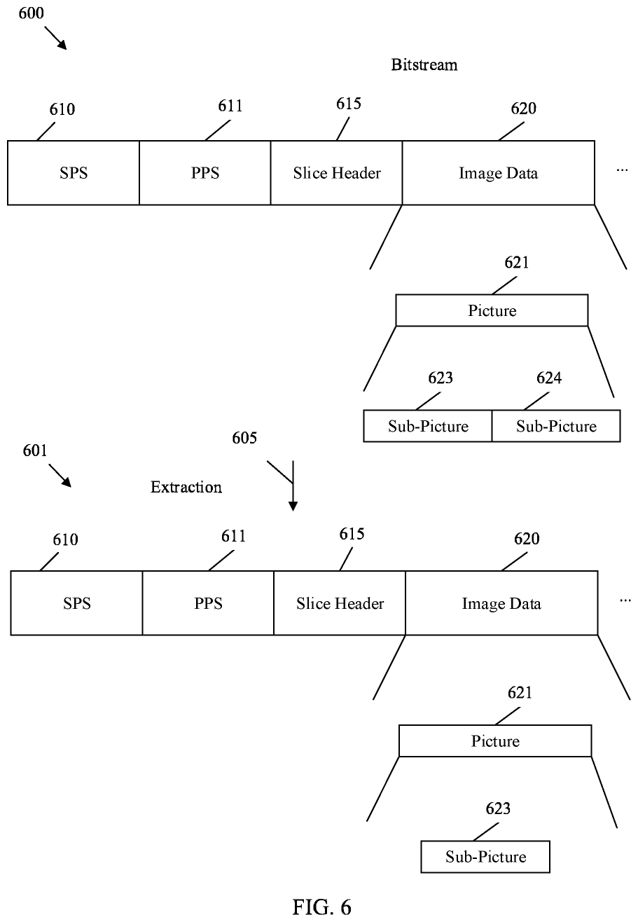

FIG. 6 is a schematic diagram illustrating an example bitstream 600 split into a sub-bitstream 601. The bitstream 600 may contain a picture video stream such as picture video stream 500, and the sub-bitstream 601 may contain a sub-picture video stream such as sub-picture video stream 501, 502, or 503. For example, the bitstream 600 and the sub-bitstream 601 can be generated by a codec system 200 or an encoder 300 for decoding by a codec system 200 or a decoder 400. As another example, the bitstream 600 and the sub-bitstream 601 may be generated by an encoder at step 109 of method 100 for use by a decoder at step 111.

The bitstream 600 includes an SPS 610, a plurality of PPSs 611, a plurality of slice headers 615, and image data 620. An SPS 610 contains sequence data common to all the pictures in the video sequence contained in the bitstream 600. Such data can include picture sizing, bit depth, coding tool parameters, or bit rate restrictions. The PPS 611 contains parameters that apply to an entire picture. Hence, each picture in the video sequence may refer to a PPS 611. While each picture refers to a PPS 611, a single PPS 611 can contain data for multiple pictures. For example, multiple similar pictures may be coded according to similar parameters. In such a case, a single PPS 611 may contain data for such similar pictures. The PPS 611 can indicate coding tools available for slices in corresponding pictures, quantization parameters, or offsets. The slice header 615 contains parameters that are specific to each slice in a picture. Hence, there may be one slice header 615 per slice in the video sequence. The slice header 615 may contain slice type information, POCs, RPLs, prediction weights, tile entry points, or deblocking parameters. A slice header 615 may also be referred to as a tile group header. A bitstream 600 may also include a picture header, which is a syntax structure that contains parameters that apply to all slices in a single picture. For this reason, a picture header and a slice header 615 may be used interchangeably. For example, certain parameters may be moved between the slice header 615 and a picture header depending on whether such parameters are common to all slices in a picture.

The image data 620 contains video data encoded according to inter-prediction, intra-prediction, or inter-layer prediction, as well as corresponding transformed and quantized residual data. For example, a video sequence includes a plurality of pictures 621. A picture 621 is an array of luma samples or an array of chroma samples that create a frame or a field thereof. A frame is a complete image that is intended for complete or partial display to a user at a corresponding instant in a video sequence. A picture 621 contains one or more slices. A slice may be defined as an integer number of complete tiles or an integer number of consecutive complete CTU rows (e.g., within a tile) of a picture 621 that are exclusively contained in a single NAL unit. The slices are further divided into CTUs or CTBs. A CTU is a group of samples of a predefined size that can be partitioned by a coding tree. A CTB is a subset of a CTU and contains luma components or chroma components of the CTU. The CTUs/CTBs are further divided into coding blocks based on coding trees. The coding blocks can then be encoded/decoded according to prediction mechanisms.

A picture 621 can be split into a plurality of sub-pictures 623 and 624. A sub-picture 623 or 624 is a rectangular region of one or more slices within a picture 621. Hence, each of the slices, and sub-divisions thereof, can be assigned to a sub-picture 623 or 624. This allows different regions of the picture 621 to be treated differently from a coding perspective depending on which sub-picture 623 or 624 includes such regions.

A sub-bitstream 601 can be extracted from the bitstream 600 according to a sub-bitstream extraction process 605. A sub-bitstream extraction process 605 is a specified mechanism that removes NAL units from a bitstream that are not a part of a target set resulting in an output sub-bitstream that includes the NAL units that are included in the target set. A NAL unit contains a slice. As such, the sub-bitstream extraction process 605 retains a target set of slices and removes other slices. The target set can be selected based on sub-picture boundaries. The slices in the sub-picture 623 are included in the target set and the slices in the sub-picture 624 are not included in the target set. As such, the sub-bitstream extraction process 605 creates a sub-bitstream 601 that is substantially similar to bitstream 600, but contains the sub-picture 623, while excluding the sub-picture 624. A sub-bitstream extraction process 605 may be performed by an encoder or an associated slicer configured to dynamically alter a bitstream 600 based on user behavior/requests.

Accordingly, the sub-bitstream 601 is an extracted bitstream that is a result of a sub-bitstream extraction process 605 applied to an input bitstream 600. The input bitstream 600 contains a set of sub-pictures. However, the extracted bitstream (e.g., sub-bitstream 601) contains only a subset of the sub-pictures of the input bitstream 600 to the sub-bitstream extraction process 605. The set of sub-pictures in the input bitstream 600 includes sub-pictures 623 and 624, while the sub-set of the sub-pictures in the sub-bitstream 601 includes sub-picture 623 but not sub-picture 624. Any number of sub-pictures 623-624 can be employed. For example, the bitstream 600 may include N sub-pictures 623-624 and the sub-bitstream may contain N−1 or fewer sub-pictures 623, where N is any integer value.

As described, a picture may be partitioned into multiple subpictures, wherein each subpicture covers a rectangular region and contains an integer number of complete slices. The subpicture partitioning persists across all pictures within a CVS, and the partitioning information is signaled in the SPS. A subpicture may be coded without using sample values from any other subpicture for motion compensation.

For each subpicture, a flag loop_filter_across_subpic_enabled_flag[i] specifies whether or not in-loop filtering across subpictures is allowed. The flag covers ALF, SAO, and deblocking tools. Since the value of the flag for each subpicture may be different, two adjacent subpictures may have different values of the flag. That difference affects the operation of deblocking more than ALF and SAO since deblocking changes sample values on both the left side and the right side of the boundary being deblocked. Thus, when two adjacent subpictures have different values of the flag, deblocking is not applied to samples along the boundary shared by both subpictures, resulting in visible artifacts. It is desirable to avoid those artifacts.

Disclosed herein are embodiments for filter flags for subpicture deblocking. In a first embodiment, when two subpictures are adjacent to each other (e.g., the right boundary of the first subpicture is also the left boundary of the second subpicture or the bottom boundary of the first subpicture is also the top boundary of the second subpicture), and the values of loop_filter_across_subpic_enabled_flag[i] of the two subpictures are different, two conditions apply to deblocking of the boundary shared by the two subpictures. First, for the subpicture with loop_filter_across_subpic_enabled_flag[i] equal to 0, deblocking is not applied to the blocks at the boundary shared with the adjacent subpicture. Second, for the subpicture with loop_filter_across_subpic_enabled_flag[i] equal to 1, deblocking is applied to the blocks at the boundary shared with the adjacent subpicture. To implement that deblocking, boundary strength determination is applied per the normal deblocking process, and sample filtering is applied only to samples belonging to the subpicture with loop_filter_across_subpic_enabled_flag[i] equal to 1. In a second embodiment, when there is a subpicture with the value of subpic_treated_as_pic_flag[i] equal to 1 and loop_filter_across_subpic_enabled_flag[i] equal to 0, the value of loop_filter_across_subpic_enabled_flag[i] of all subpictures shall be equal to 0. In a third embodiment, instead of signaling loop_filter_across_subpic_enabled_flag[i] for each subpicture, only one flag is signaled to specify whether or not loop filter across subpictures is enabled. The disclosed embodiments reduce or eliminate the artifacts described above and result in fewer wasted bits in the encoded bitstream.

The SPS has the following syntax and semantics to implement the embodiments.

SPS RBSP Syntax

| | Descriptor |
|---|---|
| seq_parameter_set_rbsp( ) { | |
| . . . | |
| subpics_present_flag | u(1) |
| if( subpics_present_flag ) { | |
| max_subpics_minus1 | u(8) |
| subpic_grid_col_width_minus1 | u(v) |
| subpic_grid_row_height_minus1 | u(v) |
| for( i = 0; i < NumSubPicGridRows; i++ ) | |
| for( j = 0; j < NumSubPicGridCols; j++ ) | |
| subpic_grid_idx[ i ][ j ] | u(v) |
| for( i = 0; i <= NumSubPics; i++ ) { | |
| subpic_treated_as_pic_flag[ i ] | u(1) |
| } | |
| loop_filter_across_subpic_enabled_flag | u(1) |
| } | |
| . . . | |
| } | |

As shown, instead of signaling loop_filter_across_subpic_enabled_flag[i] for each subpicture, only one flag is signaled to specify whether or not loop filter across subpictures is enabled, and that flag is signaled at the SPS level.

loop_filter_across_subpic_enabled_flag equal to 1 specifies that in-loop filtering operations may be performed across the boundaries of subpictures in each coded picture in the CVS. loop_filter_across_subpic_enabled_flag equal to 0 specifies that in-loop filtering operations are not performed across the boundaries of subpictures in each coded picture in the CVS. When not present, the value of loop_filter_across_subpic_enabled_pic_flag is inferred to be equal to 1.

### General Deblocking Filter Process

A deblocking filter is a filtering process that is applied as part of the decoding process in order to minimize the appearance of visual artefacts at the boundaries between blocks. Inputs to the general deblocking filter process are the reconstructed picture prior to deblocking (the array recPicture_{L}), and the arrays recPicture_{Cb} and recPicture_{Cr} are the inputs when ChromaArrayType is not equal to 0.

Outputs of the general deblocking filter process are the modified reconstructed picture after deblocking (the array recPicture_{L}), and the arrays recPicture_{Cb} and recPicture_{Cr} when ChromaArrayType is not equal to 0.

The vertical edges in a picture are filtered first. Then the horizontal edges in a picture are filtered with samples modified by the vertical edge filtering process as inputs. The vertical and horizontal edges in the CTBs of each CTU are processed separately on a CU basis. The vertical edges of the coding blocks in a CU are filtered starting with the edge on the left-hand side of the coding blocks proceeding through the edges towards the right-hand side of the coding blocks in their geometrical order. The horizontal edges of the coding blocks in a CU are filtered starting with the edge on the top of the coding blocks proceeding through the edges towards the bottom of the coding blocks in their geometrical order. Although the filtering process is specified on a picture basis, the filtering process can be implemented on a CU basis with an equivalent result, provided the decoder properly accounts for the processing dependency order so as to produce the same output values.

The deblocking filter process is applied to all coding subblock edges and transform block edges of a picture, except the following types of edges: edges that are at the boundary of the picture, edges that coincide with the boundaries of a subpicture when loop_filter_across_subpic_enabled_flag is equal to 0, edges that coincide with the virtual boundaries of the picture when pps_loop_filter_across_virtual_boundaries_disabled_flag is equal to 1, edges that coincide with brick boundaries when loop_filter_across_bricks_enabled_flag is equal to 0, edges that coincide with slice boundaries when loop_filter_across_slices_enabled_flag is equal to 0, edges that coincide with upper or left boundaries of slices with slice_deblocking_filter_disabled_flag equal to 1, edges within slices with slice_deblocking_filter_disabled_flag equal to 1, edges that do not correspond to 4×4 sample grid boundaries of the luma component, edges that do not correspond to 8×8 sample grid boundaries of the chroma component, edges within the luma component for which both sides of the edge have intra_bdpcm_flag equal to 1, and edges of chroma subblocks that are not edges of the associated transform unit. A subblock is a division of a block or coding block, for instance a 64×32 division of a 64×64 block. A transform block is a rectangular M×N block of samples resulting from a transform in the decoding process. A transform is a part of the decoding process by which a block of transform coefficients is converted to a block of spatial-domain values. While the deblocking filter process is discussed, the same constraints may apply to an SAO process and an ALF process.

### One-Direction Deblocking Filter Process

Inputs to the one-direction deblocking filter process are the variable treeType specifying whether the luma (DUAL_TREE_LUMA) or chroma components (DUAL_TREE_CHROMA) are currently processed, the reconstructed picture prior to deblocking (e.g., the array recPicture_{L}) when treeType is equal to DUAL_TREE_LUMA, the arrays recPicture_{Cb} and recPicture_{Cr} when ChromaArrayType is not equal to 0 and treeType is equal to DUAL_TREE_CHROMA, and a variable edgeType specifying whether a vertical (EDGE_VER) or a horizontal (EDGE_HOR) edge is filtered.

Outputs to the one-direction deblocking filter process are the modified reconstructed picture after deblocking, specifically the array recPicture_{L} when treeType is equal to DUAL_TREE_LUMA, and the arrays recPicture_{Cb} and recPicture_{Cr} when ChromaArrayType is not equal to 0 and treeType is equal to DUAL_TREE_CHROMA.

The variables firstCompIdx and lastCompIdx are derived as follows:

firstCompIdx=(treeType==DUAL_TREE_CHROMA) ? 1:0

lastCompIdx=(treeType==DUAL_TREE_LUMA∥ChromaArrayType==0) ? 0:2

For each CU and each coding block per color component of a CU indicated by the color component index cIdx ranging from firstCompIdx to lastCompIdx, inclusive, with coding block width nCbW, coding block height nCbH, and location of top-left sample of the coding block (xCb, yCb), when cIdx is equal to 0, or when cIdx is not equal to 0 and edgeType is equal to EDGE_VER and xCb % 8 is equal 0, or when cIdx is not equal to 0 and edgeType is equal to EDGE_HOR and yCb % 8 is equal to 0, the edges are filtered by the following ordered steps:

Step 1: The variable filterEdgeFlag is derived as follows: First, if edgeType is equal to EDGE_VER and one or more of the following conditions are true, filterEdgeFlag is set equal to 0: the left boundary of the current coding block is the left boundary of the picture, the left boundary of the current coding block is the left or right boundary of the subpicture and loop_filter_across_subpic_enabled_flag is equal to 0, the left boundary of the current coding block is the left boundary of the brick and loop_filter_across_bricks_enabled_flag is equal to 0, the left boundary of the current coding block is the left boundary of the slice and loop_filter_across_slices_enabled_flag is equal to 0, or the left boundary of the current coding block is one of the vertical virtual boundaries of the picture and pps_loop_filter_across_virtual_boundaries_disabled_flag is equal to 1. Second, otherwise, if edgeType is equal to EDGE_HOR and one or more of the following conditions are true, the variable filterEdgeFlag is set equal to 0: the top boundary of the current luma coding block is the top boundary of the picture, the top boundary of the current coding block is the top or bottom boundary of the subpicture and loop_filter_across_subpic_enabled_flag is equal to 0, the top boundary of the current coding block is the top boundary of the brick and loop_filter_across_bricks_enabled_flag is equal to 0, the top boundary of the current coding block is the top boundary of the slice and loop_filter_across_slices_enabled_flag is equal to 0, or the top boundary of the current coding block is one of the horizontal virtual boundaries of the picture and pps_loop_filter_across_virtual_boundaries_disabled_flag is equal to 1. Third, otherwise, filterEdgeFlag is set equal to 1. The filterEdgeFlag is a variable that specifies whether an edge of a block needs to be filtered using, for instance, in-loop filtering. An edge refers to pixels along a border of a block. A current coding block is a coding block that is currently being decoded by the decoder. A subpicture is a rectangular region of one or more slices within a picture.

Step 2: All elements of the two-dimensional (nCbW)×(nCbH) array edgeFlags, maxFilterLengthQs, and maxFilterlengthPs are initialized to be equal to zero.

Step 3: The derivation process of the transform block boundary specified in clause 8.8.3.3 of VVC is invoked with the location (xCb, yCb), the coding block width nCbW, the coding block height nCbH, the variable cIdx, the variable filterEdgeFlag, the array edgeFlags, the maximum filter length arrays maxFilterLengthPs and maxFilterLengthQs, and the variable edgeType as inputs, and the modified array edgeFlags, the modified maximum filter length arrays maxFilterLengthPs and maxFilterLengthQs as outputs.

Step 4: When cIdx is equal to 0, the derivation process of the coding subblock boundary specified in clause 8.8.3.4 of VVC is invoked with the location (xCb, yCb), the coding block width nCbW, the coding block height nCbH, the array edgeFlags, the maximum filter length arrays maxFilterLengthPs and maxFilterLengthQs, and the variable edgeType as inputs, and the modified array edgeFlags, the modified maximum filter length arrays maxFilterLengthPs and maxFilterLengthQs as outputs.

Step 5: The picture sample array recPicture is derived as follows: If cIdx is equal to 0, recPicture is set equal to the reconstructed luma picture sample array prior to deblocking recPictureL. Otherwise, if cIdx is equal to 1, recPicture is set equal to the reconstructed chroma picture sample array prior to deblocking recPictureCb. Otherwise (cIdx is equal to 2), recPicture is set equal to the reconstructed chroma picture sample array prior to deblocking recPictureCr.

Step 6: The derivation process of the boundary filtering strength specified in clause 8.8.3.5 of VVC is invoked with the picture sample array recPicture, the luma location (xCb, yCb), the coding block width nCbW, the coding block height nCbH, the variable edgeType, the variable cIdx, and the array edgeFlags as inputs, and an (nCbW)×(nCbH) array bS as an output.

Step 7: The edge filtering process for one direction is invoked for a coding block as specified in clause 8.8.3.6 of VVC with the variable edgeType, the variable cIdx, the reconstructed picture prior to deblocking recPicture, the location (xCb, yCb), the coding block width nCbW, the coding block height nCbH, and the arrays bS, maxFilterLengthPs, and maxFilterLengthQs, as inputs, and the modified reconstructed picture recPicture as output.

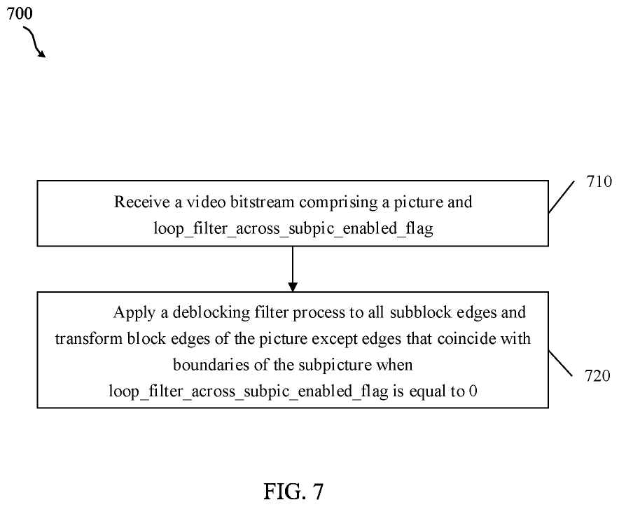

FIG. 7 is a flowchart illustrating a method 700 of decoding a bitstream according to a first embodiment. The decoder 400 may implement the method 700. At step 710, a video bitstream comprising a picture and loop_filter_across_subpic_enabled_flag is received. The picture comprises a subpicture. Finally, at step 720, a deblocking filter process is applied to all subblock edges and transform block edges of the picture except edges that coincide with boundaries of the subpicture when loop_filter_across_subpic_enabled_flag is equal to 0.

The method 700 may implement additional embodiments. For instance, loop_filter_across_subpic_enabled_flag equal to 1 specifies that in-loop filtering operations may be performed across boundaries of a subpicture in each coded picture in a CVS. loop_filter_across_subpic_enabled_flag equal to 0 specifies that in-loop filtering operations are not performed across boundaries of a subpicture in each coded picture in a CVS.

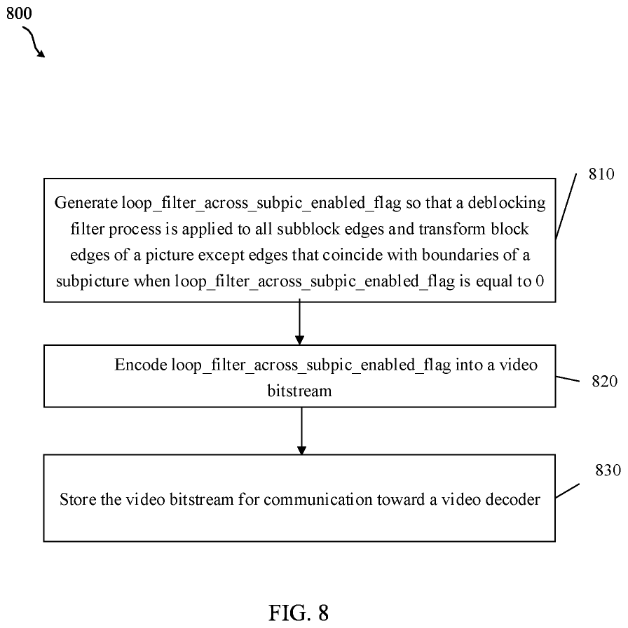

FIG. 8 is a flowchart illustrating a method 800 of encoding a bitstream according to a first embodiment. The encoder 300 may implement the method 800. At step 810, loop_filter_across_subpic_enabled_flag is generated so that a deblocking filter process is applied to all subblock edges and transform block edges of a picture except edges that coincide with boundaries of a subpicture when loop_filter_across_subpic_enabled_flag is equal to 0. At step 820, loop_filter_across_subpic_enabled_flag is encoded into a video bitstream. Finally, at step 830, the video bitstream is stored for communication toward a video decoder.

The method 800 may implement additional embodiments. For instance, loop_filter_across_subpic_enabled_flag equal to 1 specifies that in-loop filtering operations may be performed across boundaries of a subpicture in each coded picture in a CVS. loop_filter_across_subpic_enabled_flag equal to 0 specifies that in-loop filtering operations are not performed across boundaries of a subpicture in each coded picture in a CVS. The method 800 further comprises generating seq_parameter_set_rbsp, including loop_filter_across_subpic_enabled_flag in seq_parameter_set_rbsp, and further encoding loop_filter_across_subpic_enabled_flag into the video bitstream by encoding seq_parameter_set_rbsp into the video bitstream.

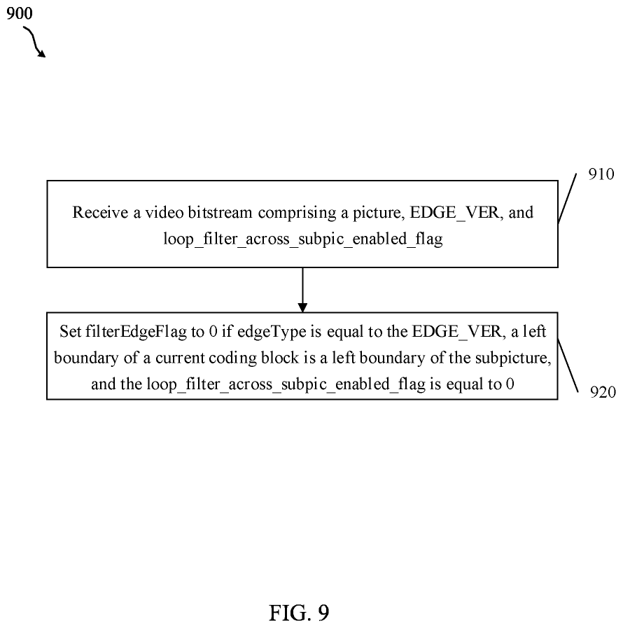

FIG. 9 is a flowchart illustrating a method 900 of decoding a bitstream according to a second embodiment. The decoder 400 may implement the method 900.

At step 910, a video bitstream comprising a picture, EDGE_VER, and loop_filter_across_subpic_enabled_flag is received. The picture comprises a subpicture. Finally, at step 920, filterEdgeFlag is set to 0 if edgeType is equal to the EDGE_VER, a left boundary of a current coding block is a left boundary of the subpicture, and the loop_filter_across_subpic_enabled_flag is equal to 0. The presence of underscores in syntax elements indicates that those syntax elements are signaled in the bitstream. The absence of underscores in syntax elements indicates derivation of those syntax elements by the decoder. “If” may also be used interchangeably with “when.”

The method 900 may implement additional embodiments. For instance, the edgeType is a variable specifying whether a vertical edge or a horizontal edge is filtered. The edgeType equal to 0 specifies that the vertical edge is filtered, and the EDGE_VER is the vertical edge. The edgeType equal to 1 specifies that the horizontal edge is filtered, and the EDGE_HOR is the horizontal edge. The loop_filter_across_subpic_enabled_flag equal to 0 specifies that in-loop filtering operations are not performed across boundaries of a subpicture in each coded picture in a CVS. The method 900 further comprises filtering the picture based on the filterEdgeFlag.

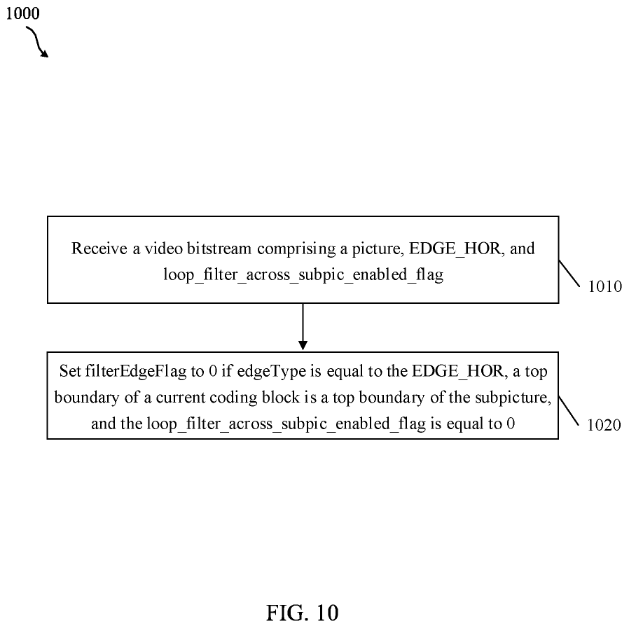

FIG. 10 is a flowchart illustrating a method 1000 of decoding a bitstream according to a third embodiment. The decoder 400 may implement the method 1000. At step 1010, a video bitstream comprising a picture, EDGE_HOR, and loop_filter_across_subpic_enabled_flag is received. Finally, at step 1020, filterEdgeFlag is set to 0 if edgeType is equal to the EDGE_HOR, a top boundary of a current coding block is a top boundary of the subpicture, and the loop_filter_across_subpic_enabled_flag is equal to 0.

The method 1000 may implement additional embodiments. For instance, the edgeType is a variable specifying whether a vertical edge or a horizontal edge is filtered. The edgeType equal to 0 specifies that the vertical edge is filtered, and the EDGE_VER is the vertical edge. The edgeType equal to 1 specifies that the horizontal edge is filtered, and the EDGE_HOR is the horizontal edge. The loop_filter_across_subpic_enabled_flag equal to 0 specifies that in-loop filtering operations are not performed across boundaries of a subpicture in each coded picture in a CVS. For instance, the method 1000 further comprises filtering the picture based on the filterEdgeFlag.

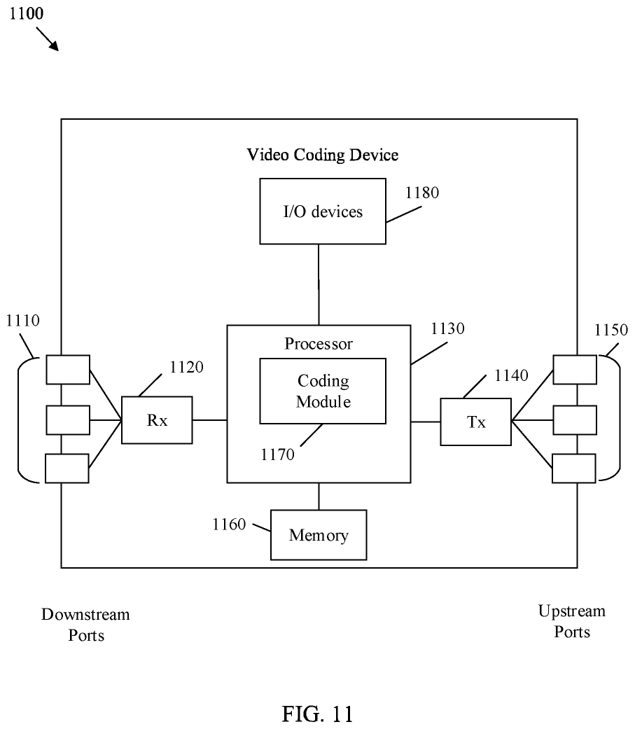

FIG. 11 is a schematic diagram of a video coding device 1100 (e.g., a video encoder 300 or a video decoder 400) according to an embodiment of the disclosure. The video coding device 1100 is suitable for implementing the disclosed embodiments. The video coding device 1100 comprises ingress ports 1110 and an Rx 1120 for receiving data; a processor, logic unit, or CPU 1130 to process the data; a Tx 1140 and egress ports 1150 for transmitting the data; and a memory 1160 for storing the data. The video coding device 1100 may also comprise OE components and EO components coupled to the ingress ports 1110, the receiver units 1120, the transmitter units 1140, and the egress ports 1150 for egress or ingress of optical or electrical signals.

The processor 1130 is implemented by hardware and software. The processor 1130 may be implemented as one or more CPU chips, cores (e.g., as a multi-core processor), FPGAs, ASICs, and DSPs. The processor 1130 is in communication with the ingress ports 1110, Rx 1120, Tx 1140, egress ports 1150, and memory 1160. The processor 1130 comprises a coding module 1170. The coding module 1170 implements the disclosed embodiments. For instance, the coding module 1170 implements, processes, prepares, or provides the various codec functions. The inclusion of the coding module 1170 therefore provides a substantial improvement to the functionality of the video coding device 1100 and effects a transformation of the video coding device 1100 to a different state. Alternatively, the coding module 1170 is implemented as instructions stored in the memory 1160 and executed by the processor 1130.

The video coding device 1100 may also include I/O devices 1180 for communicating data to and from a user. The I/O devices 1180 may include output devices such as a display for displaying video data, speakers for outputting audio data, etc. The I/O devices 1180 may also include input devices, such as a keyboard, mouse, or trackball, or corresponding interfaces for interacting with such output devices.

The memory 1160 comprises one or more disks, tape drives, and solid-state drives and may be used as an over-flow data storage device, to store programs when such programs are selected for execution, and to store instructions and data that are read during program execution. The memory 1160 may be volatile and/or non-volatile and may be ROM, RAM, TCAM, or SRAM.

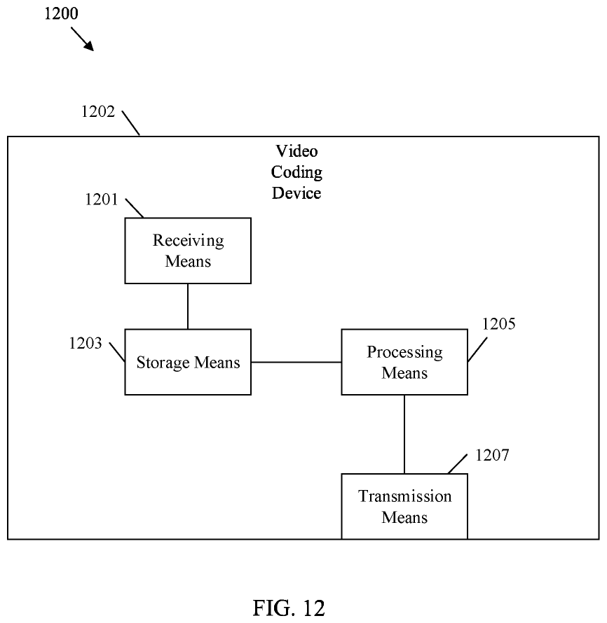

FIG. 12 is a schematic diagram of an embodiment of a means for coding 1200. In an embodiment, the means for coding 1200 is implemented in a video coding device 1202 (e.g., the video encoder 300 or the video decoder 400). The video coding device 1202 includes receiving means 1201. The receiving means 1201 is configured to receive a picture to encode or to receive a bitstream to decode. The video coding device 1202 includes transmission means 1207 coupled to the receiving means 1201. The transmission means 1207 is configured to transmit the bitstream to a decoder or to transmit a decoded image to a display means (e.g., one of the I/O devices 1180).

The video coding device 1202 includes a storage means 1203. The storage means 1203 is coupled to at least one of the receiving means 1201 or the transmission means 1207. The storage means 1203 is configured to store instructions. The video coding device 1202 also includes processing means 12305. The processing means 1205 is coupled to the storage means 1203. The processing means 1205 is configured to execute the instructions stored in the storage means 1203 to perform the methods disclosed herein.

In an embodiment, a receiving means receives a video bitstream comprising a picture and loop_filter_across_subpic_enabled_flag. The picture comprises a subpicture. A processing means applies a deblocking filter process to all subblock edges and transform block edges of the picture except edges that coincide with boundaries of the subpicture when loop_filter_across_subpic_enabled_flag is equal to 0.

The term “about” means a range including ±10% of the subsequent number unless otherwise stated. While several embodiments have been provided in the present disclosure, it may be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, components, techniques, or methods without departing from the scope of the present disclosure. Other items shown or discussed as coupled may be directly coupled or may be indirectly coupled or communicating through some interface, device, or intermediate component whether electrically, mechanically, or otherwise. Other examples of changes, substitutions, and alterations are ascertainable by one skilled in the art and may be made without departing from the spirit and scope disclosed herein.

What is claimed is:

1. A method implemented by a video decoder and comprising:
receiving, by the video decoder, a video bitstream comprising a picture and loop_filter_across_subpic_enabled_flag, wherein the picture comprises a subpicture; and
applying a deblocking filter process to all subblock edges and transform block edges of the picture except edges that coincide with boundaries of the subpicture when loop_filter_across_subpic_enabled_flag is equal to 0.

2. The method of claim 1, wherein loop_filter_across_subpic_enabled_flag equal to 1 specifies that in-loop filtering operations may be performed across boundaries of a subpicture in each coded picture in a coded video sequence (CVS).

3. The method of claim 1, wherein loop_filter_across_subpic_enabled_flag equal to 0 specifies that in-loop filtering operations are not performed across boundaries of a subpicture in each coded picture in a coded video sequence (CVS).

4. A video decoder comprising:
a memory configured to store instructions; and
a processor coupled to the memory and configured to execute the instructions to cause the video decoder to:
receive a video bitstream comprising a picture and loop_filter_across_subpic_enabled_flag, wherein the picture comprises a subpicture; and
apply a deblocking filter process to all subblock edges and transform block edges of the picture except edges that coincide with boundaries of the subpicture when loop_filter_across_subpic_enabled_flag is equal to 0.

5. A method implemented by a video decoder and comprising:
receiving, by the video decoder, a video bitstream comprising a picture and loop_filter_across_subpic_enabled_flag, wherein the picture comprises a subpicture; and
setting filterEdgeFlag to 0 if edgeType is equal to EDGE_VER, a left boundary of a current coding block is a left boundary of the subpicture, and the loop_filter_across_subpic_enabled_flag is equal to 0.

6. The method of claim 5, wherein the edgeType is a variable specifying whether a vertical edge or a horizontal edge is filtered.

7. The method of claim 6, wherein the edgeType equal to 0 specifies that the vertical edge is filtered.

8. The method of claim 6, wherein the EDGE_VER is the vertical edge.

9. The method of claim 6, wherein the edgeType equal to 1 specifies that the horizontal edge is filtered.

10. The method of claim 5, wherein the loop_filter_across_subpic_enabled_flag equal to 0 specifies that in-loop filtering operations are not performed across boundaries of a subpicture in each coded picture in a coded video sequence (CVS).

11. The method of claim 5, further comprising filtering the picture based on the filterEdgeFlag.

12. A video decoder comprising:
a memory configured to store instructions; and
a processor coupled to the memory and configured to execute the instructions to cause the video decoder to:
receive a video bitstream comprising a picture and loop_filter_across_subpic_enabled_flag, wherein the picture comprises a subpicture; and
set filterEdgeFlag to 0 if edgeType is equal to EDGE_VER, a left boundary of a current coding block is a left boundary of the subpicture, and the loop_filter_across_subpic_enabled_flag is equal to 0.

13. A method implemented by a video decoder and comprising:
receiving, by the video decoder, a video bitstream comprising a picture and loop_filter_across_subpic_enabled_flag, wherein the picture comprises a subpicture; and
setting filterEdgeFlag to 0 if edgeType is equal to EDGE_HOR, a top boundary of a current coding block is a top boundary of the subpicture, and the loop_filter_across_subpic_enabled_flag is equal to 0.

14. The method of claim 13, wherein the edgeType is a variable specifying whether a vertical edge or a horizontal edge is filtered.

15. The method of claim 14, wherein the edgeType equal to 0 specifies that the vertical edge is filtered.

16. The method of claim 14, wherein the edgeType equal to 1 specifies that the horizontal edge is filtered.

17. The method of claim 14, wherein the EDGE_HOR is the horizontal edge.

18. The method of claim 13, wherein the loop_filter_across_subpic_enabled_flag equal to 0 specifies that in-loop filtering operations are not performed across boundaries of a subpicture in each coded picture in a coded video sequence (CVS).

19. The method of claim 13, further comprising filtering the picture based on the filterEdgeFlag.

20. A video decoder comprising:
a memory configured to store instructions; and
a processor coupled to the memory and configured to execute the instructions to cause the video decoder to:
receive, by the video decoder, a video bitstream comprising a picture and loop_filter_across_subpic_enabled_flag, wherein the picture comprises a subpicture; and
set filterEdgeFlag to 0 if edgeType is equal to EDGE_HOR, a top boundary of a current coding block is a top boundary of the subpicture, and the loop_filter_across_subpic_enabled_flag is equal to 0.
